(19) United States
(12) Patent Application Publication
(10) Pub. No.: US 2009/0192226 A1
(43) Pub. Date: Jul. 30, 2009

(54) Materials and Methods for Treatment of Pathological Ocular Vascular Proliferation

(76) Inventors: Josef Neu, Gainesville, FL (US); Maria B. Grant, Fairfield, FL (US); Joshua Anthony, Evansville, IN (US); Kristin Morris, Evansville, IN (US); Eduard Poels, Newburgh, IN (US); Deborah Schade, Evansville, IN (US); Hugh Tucker, Evansville, IN (US)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/336,814

(22) Filed: Dec. 17, 2008

Related U.S. Application Data
(60) Provisional application No. 61/014,180, filed on Dec. 17, 2007.

(51) Int. Cl. A61K 31/202; A61P 35/00

(52) U.S. Cl. 514/560

(57) ABSTRACT

The subject invention provides for the administration of docosahexaenoic acid in preventing pathological proliferation of blood vessels. The compositions of the subject invention are particularly advantageous because they are stable, bioavailable, and can be formulated in an aqueous solution.

### CROSS-REFERENCE TO A RELATED APPLICATION

This application claims the benefit of U.S. provisional application Ser. No. 61/014,180, filed Dec. 17, 2007, incorporated herein by reference in its entirety.

### BACKGROUND OF THE INVENTION

People suffering from visual impairment face many challenges in performing routine daily activities and/or may not be able to fully enjoy the visual aspects of their surroundings. Of particular concern with regard to the current invention are visual impairments caused by damage to the retina, which occur in conditions such as diabetic retinopathy and retinopathy of prematurity.

Diabetic retinopathy is a progressive disease characterized by abnormalities of the blood vessels of the retina caused by diabetes, such as weakening of the blood vessel walls, leakage from the blood vessels, and bleeding and scarring around new vessels. Diabetic retinopathy results in impairment of a person's vision causing severely blurred vision and, potentially, blindness.

Diabetes affects over 16 million Americans. The World Health Organization indicates that diabetes afflicts 120 million people worldwide, and estimates that this number will increase to 300 million by the year 2025. Diabetics are faced with numerous complications including kidney failure, non-traumatic amputations, an increase in the incidence of heart attack or stroke, nerve damage, and loss of vision. Diabetic retinopathy is a form of visual impairment often suffered by diabetics.

Due to significant medical advancements, diabetics are able to live much longer than in the past. However, the longer a person has diabetes the greater the chances of developing diabetic retinopathy. Affecting over 5.3 million Americans, diabetic retinopathy is the leading cause of blindness among adults in the United States. Annually, in the United States, between 12,000 and 24,000 people lose their sight because of diabetes.

While management of diabetic retinopathy has improved, risk of complications, such as loss of visual acuity, loss of night vision and loss of peripheral vision, remains significant and treatment sometimes fails. Currently, laser photocoagulation is the most effective form of therapy for advanced disease. Unfortunately, current treatment options are inadequate and the disease is often progressive even with successful glucose control.

Retinopathy of prematurity (ROP) is a disorder of retinal blood vessel development in the premature infant. Under normal development, blood vessels grow from the back central part of the eye out toward the edges. In premature babies, this process is not complete and the abnormal growth of the vessels proliferate leading to scar tissue development, retinal detachment and possibly complete blindness.

ROP is the major cause of blindness in children under the age of 7. The salient pathological features are neovascularization in the retinal vascular endothelium with edema and breakdown in the blood-retinal barrier (BRB) that leads to hemorrhage, tissue damage and retinal scarring ultimately leads, in the severest cases, to blindness.

Improved care in the neonatal intensive care unit has reduced the incidence of retinopathy of prematurity in moderately premature infants. Ironically, however, increasing rates of survival of very premature infants, who would have had little chance of survival in the past, has increased the occurrence of retinopathy of prematurity. Since these very premature infants are at the highest risk of developing ROP, it is of great concern that the condition may actually be becoming more prevalent again.

For those babies in whom retinopathy progresses, treatment is necessary. Cryotherapy and laser treatment have some effect in advanced stages of the disease, saving a degree of vision in a proportion of the eyes that would otherwise have been blinded, but prevention awaits a better understanding of major causative factors and underlying pathophysiology.

Current research shows promise that the prevention of retinal blood vessel damage, which marks retinopathy, may be achieved by the utilization of certain compounds. It has been demonstrated that, in retinal epithelial cells, glutamine deprivation can lead to upregulation of vascular endothelial growth factor (VEGF) expression (Abcouwer S. et al., “Response of VEGF expression to amino acid deprivation and inducers of endoplasmic reticulum stress”, Invest Opthalmol Vis Sci, August 2002, pp. 2791-8, Vol. 43, No. 8). Most sick premature infants are deprived of glutamine during the time they receive supplemental oxygen, a known predisposing factor in the development of ROP. The over expression of VEGF during this time period is also thought to be involved in the pathogenesis of ROP providing glutamine supplements during this time period could potentially down-regulate VEGF. Arginine is substrate for the reaction that produces nitric oxide, a very potent vasodilator, vasodilation in retinal blood vessels also prevents neovascularization. Nitirc oxide also has numerous other beneficial effects and is now commonly used for treatment of lung disease in critically ill infants.

It is well known that proteins are converted to amino acids in the digestive system and that the resulting amino acids are used by the body for growth and development. Proteins and peptides administered for therapeutic or preventative measures are also well-known. Oligopeptides are better absorbed in the intestines than individual amino acids.

Experiments involving the use of total parenteral nutrition (TPN) containing glycyl-glutamine dipeptides, however, suggest potential adverse effects of the TPN formulation containing glycyl-glutamine (U.S. Pat. No. 5,189,016).

Also, the use of an arginyl-glutamine dipeptide for the prevention of muscle breakdown, microbial infections, and pathological vascular proliferation has been described. See, WO 03/017787 and WO 05/030242. These amino acids have also been described in complex compositions (Miyazawa et al. (1976) Journal of Faculty of Fisheries and Animal Husbandry Hiroshima 15(2): 161-169; and JP 2119762).

With the increase of adult onset diabetes, longer life span for diabetics and high rate of survival of very premature infants, many individuals are now at even greater risk for developing retinopathy. Although treatment options, such as laser therapy, exist for both conditions, the results are inadequate and the disease often remains progressive. There remains a great need in the art for compositions which prevent retinal diseases.

### BRIEF SUMMARY OF THE INVENTION

The subject invention provides materials and methods useful in preventing pathological proliferation of blood vessels. The prevention of the over-proliferation of these blood vessels according to the subject invention is particularly advantageous for treatment of certain ocular conditions including treating premature infants at risk for retinopathy of prematurity and individuals at risk for diabetic retinopathy.

Specifically exemplified herein is the use of a decosahexaenoic acid (DHA) to treat ocular disorders. In a specific examples a neonate is treated with a composition comprising DHA in order to provide beneficial effects in a safe, easily absorbable formulation.

In one embodiment of the subject invention, DHA is administered together with arginine and glutamine.

Advantageously, the composition and methods of the subject invention inhibit the over-proliferation of unwanted blood vessels. The composition of the subject invention is also advantageous because it is safe for human and animal use and can be readily formulated in an aqueous solution.

The compounds of the subject invention can be formulated according to known methods for preparing pharmaceutically useful compositions. In general, the compositions of the subject invention will be formulated such that an effective amount of the bioactive compound(s) is combined with a suitable carrier in order to facilitate effective administration of the composition.

The subject invention provides pharmaceutical compositions comprising, as an active ingredient, an effective amount of DHA, or a salt thereof, and one or more non-toxic, pharmaceutically acceptable carriers or diluents. Pharmaceutical carriers or excipients may contain inert ingredients which do not interact with the compound, or ingredients that do interact with the compound but not in a fashion so as to interfere with the desired effect. In general, the formulations are prepared by uniformly and intimately bringing into association the active ingredient with liquid carriers or finely divided solid carriers or both, and then, if necessary, shaping the product. Examples of such carriers for use in the invention include ethanol, dimethyl sulfoxide, glycerol, silica, alumina, starch, and equivalent carriers and diluents.

### DETAILED DISCLOSURE OF THE INVENTION

The present invention provides compositions containing therapeutic compounds and methods for administering the same. In one embodiment, the subject invention provides a novel, safe and affordable therapy for treatment of pathological ocular vascular proliferation.

The subject invention comprises administering an omega 3 fatty acid, especially a long chain polyunsaturated fatty acid (PUFA), such as DHA. In a further embodiment, the methods of the subject invention include the administration of arachidonic acid (AA).

The subject invention contemplates the administration of the DHA in any appropriate formulation including, for example, salts, and extended release formulations (such as, for example, formulation with polyethylene glycol (PEG)).

Advantageously, the subject invention provides a composition having excellent water solubility, stability to sterilization, long-term stability, and bioavailability for humans and animals. One embodiment of the present invention provides a composition comprising an aqueous pharmaceutical solution having DHA and arginine and glutamine.

The invention described herein contemplates the administration of arginine and glutamine in any form that can be ingested and absorbed by a subject. In one embodiment, arginine and glutamine are administered as free amino acids or salts, precursors, and/or prodrugs thereof. In a specific embodiment, the arginine and glutamine are administered as free arginine and free glutamine.

In another embodiment, the arginine and glutamine are administered in the form of the dipeptide arginyl-glutamine. In this embodiment, the solubility of the dipeptide may be greater than the solubility of the individual amino acids.

In a further embodiment, the arginine and glutamine are administered as an alanyl-glutamine dipeptide and free arginine. In yet another embodiment, the arginine and glutamine are administered as a glutamine-glutamine dipeptide and free arginine. In a particular embodiment, the arginine and glutamine are administered as a glycyl-glutamine dipeptide and free arginine.

Any synthetic or naturally-occurring dipeptide, tripeptide, or other small oligopeptide, containing or otherwise enriched with arginine and glutamine, may be used in the practice of the invention, provided the formulation comprises an efficacious amount of arginine and glutamine for the intended benefit. The selection of the particular form of arginine and glutamine depends upon the particular use for the formulation. For example, the administration of an arginine-glutamine dipeptide, rather than administration of the free amino acids, permits administration of the same amount of amino acid residue in solutions which are less hypertonic and, therefore, of lower osmolality.

In a still further embodiment, proteins or protein hydrolysates may serve as a source of the arginine and glutamine. Examples of sources for arginine and glutamine include peptides of polyarginine and polyglutamine, peptides containing blocks of polyarginine and polyglutamine, and peptides of alternating arginine and glutamine. In the case of oligopeptides, peptides, and proteins that contain the arginine-glutamine dipeptide, these prodrug formulations may be designed with, for example, cleavage sites adjacent to each side of the arginine-glutamine dipeptide so that the dipeptide is generated upon exposure to enzymes, acids, or other factors.

In one embodiment, a polypeptide can be prepared with multiple arginine-glutamine dipeptides separated by cleavage sites. When the polypeptide is exposed to a cleaving factor, which breaks apart the polypeptide, it is separated into multiple arginine-glutamine dipeptides. This cleaving to create the dipeptide can be performed as part of a production process or in vivo as the result of, for example, digestive enzymes and/or acids.

If the arginine-glutamine dipeptide is administered in the form of a prodrug, in some embodiments, the prodrug can be converted to a biologically active compound at a controlled rate via passive (such as by aqueous hydrolysis) mechanisms or biologically-mediated (such as biocatalytic or enzymatic) mechanisms. In this embodiment, the in vivo conversion of the prodrug may provide localized therapeutic effects in target disease tissue with high therapeutic margins of safety.

In some embodiments, the arginine-glutamine dipeptide results in minimal cyclisation of glutamine into pyro-glutamate. In one particular embodiment, the arginine-glutamine dipeptide of the invention has an N-terminal amino acid, which is arginine, and a C-terminal amino acid, which is glutamine.

If provided as an arginine-glutamine dipeptide, the arginine-glutamine dipeptide can be readily synthesized and/or formulated by a person skilled in the art having the benefit of the present disclosure. Alternatively, the dipeptides can be purchased commercially from, for example, Bachem Biosciences, Inc., which sells an arginine-glutamine dipeptide salt. DIPEPTIVEN™ is available from Fresenius Kabi, Uppsala, Sweden, and is a 20% solution of N(2)-L-alanyl-glutamine. Further information is found in Fürst et al., The J. of Nutrition (Suppl): 2562S-2568S (2001). If used, the arginine-glutamine dipeptide can be of any purity or grade, and can be of a purity and grade that is suitable for inclusion in the diet of the subject.

Unless the context dictates otherwise as used herein, the term “comprising” contemplates the optional circumstances of “consisting of” and “consisting essentially of.”

In a specific embodiment of the subject invention the compositions described herein can be used for preventing the proliferation of abnormal retinal blood vessels in a patient. Thus, these compositions can be administered to premature infants or diabetics who are at risk for retinal disease. Enteral and parenteral formulations are contemplated.

As discussed in more detail below, in addition to DHA and, optionally the arginine and glutamine and/or AA, the clinical solution of the subject invention can contain, for example, dextrose, liquid emulsions, vitamins, minerals, trace elements, and other components. The selection of the particular amino acid formulation depends upon the particular use. The concentration of the total amount of arginine and glutamine in the aqueous solution can be, for example, from about 0.1 to about 25.0 percent by weight. The concentration may also be between, for example, 0.1% and 10%, or 0.2% and 5%.

For parenteral administration, a supply of the solution may be merged through a Y-connection with a supply of glucose solution or other parenteral solutions. The solutions may also be mixed with glucose solutions and/or other parenteral solutions to create a mixture which may be administered parenterally.

In one method, the subject invention involves identifying an individual who has, or who is at risk for developing, pathological vascularization and then providing that individual with a composition comprising DHA according to the subject invention along with instructions or information concerning the activity of DHA to inhibit pathological vascularization.

The compositions of the invention are useful for various therapeutic purposes. Specifically, as described herein, the compounds of the invention are effective for inhibiting vascular retinopathy and other forms of pathological vascular proliferation. Accordingly, these compounds are useful prophylactically and therapeutically for treating animals, including humans and other mammals, at risk for pathological vascular proliferation including vascular retinopathy and vasculature associated with tumors.

Therapeutic application of the compounds and compositions containing them can be accomplished by any suitable therapeutic method and technique presently or prospectively known to those skilled in the art.

The compositions provided by the present invention are typically administered to a mammal, particularly a human, dog or cat, any of which is intended to be encompassed by the term “patient” herein, in need of the prevention or treatment of pathological vascular proliferation. Pathological conditions involving vascular proliferation include, for example, tumor growth, age-related macular degeneration, vascular proliferation associated with angioplasty and/or stents, diabetic retinopathy and retinopathy of prematurity. Thus, DHA can be used to treat angiogenic diseases. Angiogenic diseases include those that are disclosed in U.S. Pat. No. 5,759,547, which is incorporated herein, in its entirety, by reference.

The compositions are administered by incorporating the DHA into a pharmaceutical composition optionally comprising arginine and glutamine or a non-toxic pharmaceutically acceptable salt and a non-toxic pharmaceutically acceptable carrier thereof.

The DHA is employed in an effective amount i.e. an amount sufficient to evoke the desired pharmacological response. This is generally an amount sufficient to produce lessening of one or more of the effects of pathological vascular proliferation. In the case of retinopathy, it is an amount sufficient to produce regression of neovascularization and/or an amount sufficient to produce improved visual acuity.

The amount of DHA administered according to the invention may be from about 3 mg per kg of body weight per day to about 150 mg per kg of body weight per day. In one embodiment of the invention, the amount is from about 6 mg per kg of body weight per day to about 100 mg per kg of body weight per day. In another embodiment the amount is from about 15 mg per kg of body weight per day to about 60 mg per kg of body weight per day. In another embodiment of the invention, the amount is from about 102 mg per kg of body weight per day to about 206 mg per kg of body weight per day. In still another embodiment, the amount is about 20 mg per kg of body weight per day. In a particular embodiment, the amount is about 50 mg per kg of body weight per day. In yet another embodiment, the amount is about 17 mg per kg of body weight per day.

If administered as part of the invention, a single dosage of the inventive composition may contain from about 90 mg per day to about 180 mg per day of DHA. In another embodiment, a single dosage of the inventive composition may contain from about 100 mg per day to about 200 mg per day of DHA.

If administered as part of the present invention, the total amount of arginine and glutamine administered may be from about 50 mg per kg of body weight per day to about 1000 mg per kg body weight per day. In another embodiment, the total amount of arginine and glutamine administered may be from about 375 mg per kg of body weight per day to about 750 mg per kg body weight per day. In another embodiment, the total amount of arginine and glutamine administered may be from about 62.5 mg per kg of body weight per day to about 125 mg per kg body weight per day.

If administered as part of the invention, a single dosage of the inventive composition may contain from about 110 mg per day to about 220 mg per day of total arginine and glutamine. In another embodiment, a single dosage of the inventive composition may contain from about 125 mg per day to about 250 mg per day of total arginine and glutamine.

If administered as part of the present invention, the amount of AA administered may be from about 5 mg per kg of body weight per day to about 150 mg per kg of body weight per day. In one embodiment of this invention, the amount varies from about 10 mg per kg of body weight per day to about 120 mg per kg of body weight per day. In another embodiment, the amount varies from about 15 mg per kg of body weight per day to about 90 mg per kg of body weight per day. In yet another embodiment, the amount varies from about 20 mg per kg of body weight per day to about 60 mg per kg of body weight per day. The terms “pharmaceutically acceptable carrier” or a “carrier” refer to any generally acceptable excipient or drug delivery device that is relatively inert and non-toxic. The DHA can be administered with or without a carrier. When treating retinopathies, one embodiment is to administer DHA to the retinal area or the vasculature around or leading to the retina. Exemplary carriers include calcium carbonate, sucrose, dextrose, mannose, albumin, starch, cellulose, silica gel, polyethylene glycol (PEG), dried skim milk, rice flour, magnesium stearate, and the like. DHA can be administered systemically or locally (e.g., by injection or diffusion). Suitable carriers (e.g., pharmaceutical carriers) also include, but are not limited to sterile water, salt solutions (such as Ringer's solution), alcohols, polyethylene glycols, gelatin, carbohydrates such as lactose, amylose or starch, magnesium stearate, talc, silicic acid, viscous paraffin, fatty acid esters, hydroxymethylcellulose, polyvinyl pyrolidone, etc. Such preparations can be sterilized and, if desired, mixed with auxiliary agents, e.g., lubricants, preservatives, stabilizers, wetting agents, emulsifiers, salts for influencing osmotic pressure, buffers, coloring, and/or aromatic substances and the like which do not deleteriously react with the active compounds. They can also be combined where desired with other active substances, e.g., enzyme inhibitors, to reduce metabolic degradation. A carrier (e.g., a pharmaceutically acceptable carrier) is preferred, but not necessary to administer the DHA.

Suitable non-toxic pharmaceutically acceptable carriers for use with the DHA and optional arginine, glutamine, or AA will be apparent to those skilled in the art of pharmaceutical formulation. See, for example, Remington's Pharmaceutical Sciences, seventeenth edition, ed. Alfonso R. Gennaro, Mack Publishing Company, Easton, Pa. (1985). The choice of suitable carriers will depend upon the exact nature of the particular dosage form selected.

The supplement can take on various forms, including but not limited to pills, edible bars, drinks or drink mix. The compounds of the subject invention may be combined with other components such as, for example, a soluble fiber compound. The soluble fiber compound may be, for example, locust gum, guar gum, pectin, gum arabic, or psyllium.

The person skilled in this art, having the benefit of the current disclosure can readily formulate the compounds of the subject invention into a pill, bar, or other edible composition for easy and enjoyable consumption. These therapeutic compositions can be used as described herein. In one embodiment, the DHA of the subject invention can be administered as a nutriceutical supplement in unit dosage form.

Therapeutic application of the new compositions can be accomplished by any suitable therapeutic method and technique presently or prospectively known to those skilled in the art.

The therapeutic dosage range can be determined by one skilled in the art having the benefit of the current disclosure. Naturally, such therapeutic dosage ranges will vary with the size, species and physical condition of the patient, the severity of the patient's medical condition, the particular dosage form employed, the route of administration and the like. In addition, a route of administration may be selected to slowly release the chemical, e.g., slow intravenous infusion.

One embodiment of the current invention envisions parenteral administration, especially intravenous administration, as the route of administration. Parenteral dosage forms should be sterile and pyrogen-free, and are prepared in accord with accepted pharmaceutical procedures. The parenteral formulations may be organic or aqueous or mixed organic/aqueous formulations and may further contain anti-oxidants, buffers, bacteriostats, isotonicity adjusters and like additions acceptable for parenteral formulations.

For parenteral application, particularly suitable are injectable, sterile solutions, preferably oily or aqueous solutions, as well as suspensions, emulsions, or implants. In particular, carriers for parenteral administration include aqueous solutions of dextrose, saline, pure water, ethanol, glycerol, propylene glycol, peanut oil, sesame oil, polyoxyethylene-polyoxypropylene block polymers, and the like. Ampules are convenient unit dosages.

Also, according to the subject invention, the local administration of the compounds, and formulations thereof, by means of a drug delivery device or implant placed in proximity to the local tissue site provides for the maintenance of efficacious, safe levels of active drug ingredient at the local tissue disease site.

According to the subject invention, the local ocular administration of the compounds of the invention, and/or formulations thereof, attenuate ocular pathological disease processes. Thus, local ocular administration of a compound of the invention, and/or formulations thereof, provides for an efficacious but safe controlled concentration range of DHA directly in the eye.

Ocular therapies, as describe herein, provide significant advantages for treating neovascular ocular disease relative to current laser surgery treatment modalities including panretinal photocoagulation, which can be accompanied by extensive ocular tissue damage. In the examples of posterior neovascular ocular diseases, such as age related macular degeneration and diabetic retinopathy, target ocular pathologies and tissues for treatment are especially localized to the retinal, choroidal and corneal ocular compartments.

The DHA can be administered locally to the eye, retinal area, choroid area or associated vasculature. The composition can also be administered to the cornea of the eye. The composition diffuses into the eye and contacts the retina or surrounding vasculature (e.g., eye drops, creams or gels).

The compositions of the present invention, and formulations thereof, are advantageous because they overcome problems associated with stability, toxicity, lack of target tissue specificity, safety, efficacy, extent and variability of bioavailability.

A further embodiment of the subject invention provides for the local administration of DHA in combination with other pharmacological therapies. As contemplated in the subject invention, combination therapies with other medicaments targeting similar or distinct disease mechanisms have advantages of greater efficacy and safety relative to respective monotherapies with either specific medicament.

In one embodiment, DHA is used to treat neovascular ocular disease by localized (for example, in ocular tissue) concurrent administration with other medicaments that act to block angiogenesis by pharmacological mechanisms. Medicaments that can be concurrently administered with DHA include, but are not limited to, vascular endothelial growth factor VEGF blockers (e.g. by VEGF neutralizing binding molecules such as Macugen (Eyetech) and Lucentis (ranibizumab, Genentech), Squalamine lactate (Genaera Corporation); and VEGF tyrosine kinase inhibition) for treating neovascular ocular disease (AMD and Diabetic Retinopathy) and glucocorticoids (e.g. Triamcinolone) for treating macular edema.

One or more active agents can be administered. When administering more than one, the administration of the agents can occur simultaneously or sequentially in time. The agents can be administered before and after one another, or at the same time. The methods also include co-administration with other drugs that are used to treat retinopathy or other diseases described herein.

The composition can be administered in a single dose or in more than one dose over a period of time to confer the desired effect.

The dosage administration to a host in the above indications will be dependent upon the specific condition being treated, the type of host involved, its age, weight, health, kind of concurrent treatment, if any, frequency of treatment, and therapeutic ratio. Those skilled in the art will be able to determine the appropriate dosages depending on these and other factors.

To provide for the administration of such dosages for the desired therapeutic treatment, new pharmaceutical compositions of the invention may comprise between about 0.1% and 45%, and especially, 1 and 15%, by weight of the total arginine and glutamine based on the weight of the total composition including carrier or diluent.

In a retinal cell culture model used to study the effects of the arginyl-glutamine dipeptide on transepithelial resistance (TER) and vascular endothelial growth factor (VEGF), it was demonstrated that the dipeptide increased TER and decreased VEGF, both desirable effects that have been associated with a decrease in vascular proliferative retinal disease.

All patents, patent applications, provisional applications, and publications referred to or cited herein are incorporated by reference in their entirety to the extent they are not inconsistent with the explicit teachings of this specification.

Following is an example which illustrates procedures for practicing the invention. This example should not be construed as limiting. All percentages are by weight and all solvent mixture proportions are by volume unless otherwise noted.

### Example 1

Retinopathy of prematurity (ROP) is the major cause of blindness in children. Supplementation with arginine or glutamine results in improved clinical outcomes in premature infants. In this study the protective effect of oral administration of Arg-Gln alone and in combination with DHA was examined in pups undergoing the OIR model.

METHODS: Nursing dams and pups were returned to normal room air on P12 (postnatal day 12) and gavaged twice daily with: Arg-Gln, DHA, Arg-Gln+DHA or vehicle (P12-P17). Normoxic pups were treated in an identical manner. On P17 the pups were perfused with FITC-labeled dextran. One eye was embedded in paraffin, cross-sectioned and H&E (hematoxylin and eosin) stained for analysis of pre-retinal neovascularization. The retina from the second eye was removed and underwent microscopic analysis for the vessel regrowth, which was evaluated by vessel density.

RESULTS: The Arg-Gln dipeptide gave the greatest reduction in pre-retinal neovascularization (35±1%, P<0.001) compared to DHA (45±2%, P<0.001) or a combination of both (67±2%, P<0.001) relative to vehicle. All test compounds dramatically reduced the area of vaso-obliteration assessed in P17 pups (Arg-Gln: 4.8±1.0%, P=0.03; DHA: 3.6±1.3%, P=0.04; combination: 5.4±0.7%, P=0.02) when compared to vehicle (30.4±7.9%). Finally, intra-retinal vascular density, a measure of vascular regrowth, following hyperoxia exposure in all groups of treated pups was significantly greater than in the vehicle treated pups, but remained less than the vascular density in normoxic pups.

CONCLUSIONS: Treatment with the Arg-Gln dipeptide alone or in combination with DHA dramatically inhibited pre-retinal neovascularization, reduced vaso-obliteration and restored vascular density in the OIR mouse model.

### Example 2

### Nutritional Composition for Enteral Administration

The inventive composition may be a nutritional composition (nutritionally complete or nutritional supplement) for enteral administration. That is, it is designed for oral, intragastric, or transpyloric use. The composition of the invention may be an infant formula or adult nutritional composition that can be milk-based, soy-based, or based on other food sources. The composition may be prepared as a powder, liquid concentrate, or ready-to-use liquid nutritional composition for formulas prepared for infant, pediatric and adult populations. The inventive composition may be prepared as a nutritionally complete diet by including vitamins and minerals at acceptable levels. The compositions of the invention may provide minimal, partial, or total nutritional support. The subject composition can be in the form of a dietary product such as an infant formula, premature infant formula, human milk fortifier, food product, milk substitute, or meal replacement or supplement. As used herein, the term “infant formula” means a composition that satisfies the nutrient requirements of an infant by being a substitute for human milk. Conveniently, commercially available infant formula can be supplemented with DHA and used in the method of the invention.

In an embodiment, the composition may be a medical food that contains DHA and, optionally, arginine, glutamine, or AA. In some embodiments, the composition is an acidified product (as required by medical food regulations).

One embodiment of the invention is a dietary supplement that contains DHA and, optionally, the arginine and glutamine. AA can also be used in the methods and compositions of the subject invention. The dietary supplement is designed to be administered along with a food or nutritional composition, such as infant formula, and can either be intermixed with the food or nutritional composition prior to ingestion by the subject, or can be administered to the subject either before or after ingestion of a food or nutritional composition. The subject dietary supplement contains an amount of DHA and, optionally, the arginine and glutamine, that is effective for the prevention or treatment of retinopathy of prematurity, diabetic retinopathy, vascular proliferative retinopathy, or proliferation of abnormal vascularization, and the like.

The amount of DHA in the infant formula of the invention may be from about 2 mg/100 kcal to about 100 mg/100 kcal. In another embodiment, the amount of DHA may be from about 5 mg/100 kcal to about 75 mg/100 kcal. In yet another embodiment, the amount of DHA may be from about 15 mg/100 kcal to about 60 mg/100 kcal. In yet another embodiment, the amount of DHA may be from about 17 mg/100 kcal to about 50 mg/100 kcal. In a particular embodiment, the amount of DHA may be about 17 mg/100 kcal. In another embodiment, the amount of DHA may be about 51 mg/100 kcal. In still another embodiment, the amount of DHA may be about 34 mg/100 kcal.

If included in the infant formula, the total amount of arginine and glutamine in the infant formula may be from about 50 mg/100 kcal to about 150 mg/100 kcal. In a particular embodiment, the total amount of arginine and glutamine may be from about 62.5 mg/100 kcal to about 125 mg/100 kcal. In another embodiment, the total amount of arginine and glutamine may be from about 21 mg/100 kcal to about 42 mg/100 kcal.

If included, the amount of AA in the infant formula may be from about 4 mg/100 kcal to about 100 mg/100 kcal. In another embodiment, the amount of AA may be from about 10 mg/100 kcal to about 67 mg/100 kcal. In yet another embodiment, the amount of AA may be from about 20 mg/100 kcal to about 50 mg/100 kcal. In a particular embodiment, the amount of AA may be from about 25 mg/100 kcal to about 40 mg/100 kcal. In one embodiment, the amount of AA is about 30 mg/100 kcal.

In one embodiment, a novel infant formula containing DHA is nutritionally complete. By the term “nutritionally complete” is meant that the composition contains adequate nutrients to sustain healthy human life for extended periods. The infant formula of the invention contains ingredients which are designed to meet the nutritional needs of the human infant namely, a protein, carbohydrate and lipid source and other nutrients such as vitamins and minerals.

Besides DHA, the composition of the invention can contain a nitrogen source (i.e., amino acids and/or protein) in an amount that is typically about 1 g to about 10 g per 100 kcal of total composition, preferably about 2 g to about 6 g per 100 kcal; the amount of lipid source per 100 kcal of total composition is typically greater than 0 g up to about 6 g, preferably about 0.5 g to about 5.5 g and more preferably about 2 g to about 5.5 g; and the amount of non-fiber carbohydrate source per 100 kcal of total composition is typically about 5 g to about 20 g, preferably about 7.5 g to about 15 g. The amount of vitamins and minerals in the nutritionally complete composition is typically sufficient to meet 100% of the U.S. recommended daily intake (RDI) in about 500 to about 3,000 kcal, preferable is about 1,000 to about 3,000 kcal. In a particular embodiment, the composition may be protein-free. In such an embodiment, the composition may contain a protein equivalent source that comprises 100% free amino acids.

In one embodiment of the present nutritional composition the amount of vitamins and minerals is sufficient to meet 100% of the RDI in about 500 to about 3,000 kcal, preferably in about 1,000 to about 3,000 kcal. As used herein, the RDI's are intended to mean those published in the Federal Register, Vol. 58, No. 3, Wednesday, Jan. 6, 1993, page 2227 which are as follows: Vitamin A, 5,000 International Units; Vitamin C, 60 milligrams; Thiamin, 1.5 milligrams; Riboflavin, 1.7 milligrams; Niacin, 20 milligrams; Calcium, 1.0 gram; Iron, 18 milligrams; Vitamin D, 400 International Units; Vitamin E, 30 International Units; Vitamin B₆, 2.0 milligrams; Folic acid, 0.4 milligrams; Vitamin B₁₂, 6 micrograms; Phosphorus, 1.0 gram; Iodine, 150 micrograms; Magnesium, 400 milligrams; Zinc, 15 milligrams; Copper, 2 milligrams; Biotin, 0.3 milligram; Pantothenic acid, 10 milligrams.

In one embodiment, the novel infant formula contains total arginine and glutamine in an amount that is less than 0.1% by weight of the formula. It is preferred that the amount of arginine and glutamine in the formula is from about 0.001% to 0.098% by weight of the formula, more preferred is an amount of from about 0.01% to 0.098% by weight.

In the present method, the subject infant formula or dietary supplement is administered to an infant in an amount that is sufficient to prevent or treat retinopathy of prematurity, diabetic retinopathy, vascular proliferative retinopathy, or proliferation of abnormal vascularization.

The protein source in the composition may be any suitable protein known in the art, assuming it is compatible with the other components of the composition. The protein source may include milk protein, non-fat milk solids, whey protein, casein, soy protein, animal protein, cereal protein, vegetable protein, or combinations thereof. The protein source that is present can be non-fat milk solids, a combination of non-fat milk solids and whey protein, a partial hydrolysate of non-fat milk and/or whey solids, soy protein isolates, or partially hydrolyzed soy protein isolates. The infant formula can be casein predominant or whey predominant. The protein source may be intact, partially hydrolyzed, or extensively hydrolyzed. The protein source, in some embodiments, may be a combination of intact protein and hydrolyzed protein. The protein source may be an isolate or a concentrate. In another embodiment, the amount of protein may vary from about 1 to about 5 g/100 kcal.

The carbohydrate source in the infant formula can be any suitable carbohydrate known in the art to be suitable for use in infant formulas. Typical carbohydrate sources include sucrose, fructose, glucose, maltodextrin, lactose, corn syrup, corn syrup solids, rice syrup solids, rice starch, modified corn starch, modified tapioca starch, rice flour, soy flour, and combinations thereof. In yet another embodiment, the amount of carbohydrate may vary from about 8 to about 12 g/100 kcal.

The lipid source in the infant formula can be any lipid or fat known in the art to be suitable for use in infant formulas. Typical lipid sources include milk fat, safflower oil, egg yolk lipid, olive oil, coconut oil, palm oil, palm kernel oil, soybean oil, sunflower oil, fish oil and fractions derived thereof such as palm olein, medium chain triglycerides (MCT), and esters of fatty acids wherein the fatty acids are, for example, arachidonic acid, linoleic acid, palmitic acid, stearic acid, docosahexaenoic acid, eicosapentaenoic acid, linolenic acid, oleic acid, lauric acid, capric acid, caprylic acid, caproic acid, and the like. If utilized, the source of the long chain polyunsaturated fatty acids can be any source known in the art such as marine oil, fish oil, single cell oil, egg yolk lipid, brain lipid, and the like. The LCPUFAs can be in natural form or refined form. High oleic forms of various oils are also contemplated to be useful herein such as high oleic sunflower oil and high oleic safflower oil. Medium chain triglycerides contain higher concentrations of caprylic and capric acid than typically found in conventional oils, e.g., approximately three-fourths of the total fatty acid content is caprylic acid and one-fourth is capric acid. In an embodiment, the amount of lipid or fat may vary from about 3 to about 7 g/100 kcal.

Nutritionally complete compositions contain all vitamins and minerals understood to be essential in the daily diet and these should be present in nutritionally significant amounts. Those skilled in the art appreciate that minimum requirements have been established for certain vitamins and minerals that are known to be necessary for normal physiological function. Practitioners also understand that appropriate additional amounts (overages) of vitamin and mineral ingredients need to be provided to compensate for some loss during processing and storage of such compositions.

To select a specific vitamin or mineral compound to be used in the infant formula of the invention requires consideration of that compound's chemical nature regarding compatibility with the particular processing conditions used and shelf storage.

Examples of minerals, vitamins and other nutrients optionally present in the composition of the invention include vitamin A, vitamin B₆, vitamin B₁₂, vitamin E, vitamin K, vitamin C, folic acid, thiamine, inositol, riboflavin, niacin, biotin, pantothenic acid, choline, calcium, phosphorus, iodine, iron, magnesium, copper, zinc, manganese, chloride, potassium, sodium, selenium, chromium, molybdenum, taurine, and L-carnitine. Minerals are usually added in salt form. In addition to compatibility and stability considerations, the presence and amounts of specific minerals and other vitamins will vary somewhat depending on the intended infant population.

The infant formula of the invention also typically contains emulsifiers and stabilizers such as soy lecithin, carrageenan, and the like.

The infant formula of the invention may optionally contain other substances which may have a beneficial effect such as lactoferrin, nucleotides, nucleosides, immunoglobulins, and the like.

In some embodiments of the invention, the composition of the invention contains probiotics and/or prebiotics. The term “probiotic” means a microorganism that exerts beneficial effects on the health of the host. Any probiotic known in the art may be included in the composition, provided it is suitable for combination with the other components of the composition. For example, the probiotic may be chosen from the group consisting of Lactobacillus and Bifidobacterium. Alternatively, the probiotic can be Lactobacillus rhamnosus GG. The term “prebiotic”, as used herein, means a non-digestible food ingredient that stimulates the growth and/or activity of probiotics. In this embodiment, any prebiotic known in the art may be included in the composition, provided it is suitable for combination with the other components of the composition. In a particular embodiment, the prebiotic may be selected from the group consisting of polydextrose, fructo-oligosaccharide, gluco-oligosaccharide, galacto-oligosaccharide, inulin, isomalto-oligosaccharide, xylo-oligosaccharide, lactulose, and combinations thereof.

The infant formula of the invention is in concentrate liquid form, liquid ready to consume form, or powder form. Of course, if in powder form, the formula is diluted to normal strength with water to be in a form ready to consume.

The osmolality of the liquid infant formula of the invention (when ready to consume) is typically about 100 to 1100 mOsm/kg H₂O, more typically about 200 to 700 mOsm/kg H₂O.

The infant formula of the invention can be sterilized, if desired, by techniques known in the art, for example, heat treatment such as autoclaving or retorting, and the like.

The infant formula of the invention can be packaged in any type of container known in the art to be used for storing nutritional products such as glass, lined paperboard, plastic, coated metal cans and the like. In some embodiments, the composition is packaged via blow-fill-seal packaging techniques. In other embodiments, the composition is provided in a single dose container. The packaging of the composition may be conducted under aseptic conditions. In some embodiments, the composition is prepared such that it is acceptable for direct delivery to an infant via nasogastric tubes, nasoduodenal tubes, or nasojejunal tubes.

The infant formula of the invention is shelf stable after reconstitution. By “shelf stable” is meant that the formula in a form ready to consume remains in a single homogenous phase (i.e., does not separate into more than one phase upon visual inspection) or that the thickener does not settle out as a sediment upon visual inspection after storage overnight in the refrigerator. With the thickened nature of the product, the formula of the invention also has the advantage of remaining fluid (i.e., does not gel into a solid mass when stored overnight in the refrigerator).

In the method of the invention, infant formula comprising DHA and, optionally, arginine and glutamine is administered to an infant. The form of administration is oral, which includes tube feeding.

The invention provides a commercially acceptable product in terms of desired stability and physical characteristics and the product demonstrates little to no observable browning effect by-products associated with a Maillard reaction. Further, the inventive composition is substantially homogeneous for an acceptable period after reconstitution (or for the shelf-life if prepared as a liquid). The invention is particularly useful for infant formula preparations for the prevention and treatment of retinopathy of prematurity, although it is equally applicable to other elemental diets specific to a selected population that is at risk of, or is suspected of having, diabetic retinopathy, vascular proliferative retinopathy, or proliferation of abnormal vascularization, and the like.

It should be understood that the examples and embodiments described herein are for illustrative purposes only and that various modifications or changes in light thereof will be suggested to persons skilled in the art and are to be included within the spirit and purview of this application.

We claim:

1. A method for inhibiting pathological vascular proliferation wherein the method comprises administering, to a patient in need of such inhibition, DHA.

2. The method, according to claim 1, used to treat pathological ocular vascular proliferation.

3. The method, according to claim 2, wherein the vascular proliferation is associated with retinopathy of prematurity or diabetic retinopathy.

4. The method, according to claim 1, wherein the vascular proliferation being inhibited is in a mammal.

5. The method, according to claim 1, which further comprises administration of arginine and glutamine, or salts thereof.

6. The method, according to claim 1, which comprises systemic administration of the DHA.

7. The method, according to claim 1, wherein the administration is enteral.

8. The method, according to claim 1, wherein said DHA is administered in an aqueous formulation.

9. The method, according to claim 4, wherein said mammal is a human.

10. The method, according to claim 1, which further comprises the administration of AA.

11. A pharmaceutical composition consisting essentially of DHA and Arg-Gln, or a salt thereof, and a pharmaceutically acceptable carrier.

12. A formulation for enteral administration for the prevention or treatment of a condition that is selected from retinopathy of prematurity, diabetic retinopathy, vascular proliferative retinopathy, or proliferation of abnormal vascularization, where the formulation comprises DHA.

13. The formulation according to claim 12, further comprising arginine and glutamine.

14. An infant formula comprising a protein source, a fat source, a carbohydrate source, DHA, arginine, and glutamine.

15. The infant formula of claim 14 wherein the arginine and glutamine are in the form of an arginine-glutamine dipeptide.

16. The infant formula of claim 14 additionally comprising AA.

17. The infant formula of claim 14 wherein DHA is present in an amount of from about 15 mg/100 kcal to about 60 mg/100 kcal.

18. The infant formula of claim 14 wherein DHA is present in an amount of from about 17 mg/100 kcal to about 50 mg/100 kcal.

19. The infant formula of claim 14 wherein the total amount of arginine and glutamine is from about 21 mg/100 kcal to about 42 mg/100 kcal.

20. The infant formula of claim 14 wherein the total amount of arginine and glutamine is from about 62.5 mg/100 kcal to about 125 mg/100 kcal.
